(19) United States
(12) Patent Application Publication
(10) Pub. No.: US 2017/0224252 A1
(43) Pub. Date: Aug. 10, 2017

(54) Dentally-Based Concussion Sensing System for Enhanced Detection of Brain Injuries

(71) Applicant: University of Virginia Patent Foundation, Charlottesville, VA (US)

(72) Inventors: Robert S. Salzar, Charlottesville, VA (US); Thomas Frank Hady, Portland, OR (US); Andrew Gray Heppe, Spotsylvania, VA (US)

(73) Assignee: University of Virginia Patent Foundation, Charlottesville, VA (US)

(21) Appl. No.: 15/386,076

(22) Filed: Dec. 21, 2016

Related U.S. Application Data
(60) Provisional application No. 62/292,051, filed on Feb. 5, 2016.
(60) Provisional application No. 62/336,899, filed on May 16, 2016.

(51) Int. Cl. A61B 5/11; A61B 5/00

(52) U.S. Cl. CPC A61B 5/1104; A61B 5/7275; A61B 5/4064; A61B 5/6803; A61B 5/6802; A61B 2503/10; A61B 2560/0219

(57) ABSTRACT

A system and related devices for continuous and real-time monitoring and detection of head impact and corresponding trauma. The system may include a passive sensor mounted to a subject's tooth using dental hardware or adhesive so as to harness the firm coupling between the subject's tooth and cranium. With such firm coupling established, the impact data measured by the tooth-mounted sensor can be manipulated, through the use of a transfer function, by external processors to determine and communicate the impact experienced by the subject's head. The system is configured for use with an external power source whereby the passive tooth-mounted sensor is activated by means of a wireless transfer circuit such as an inductive power transfer circuit or an ultrasound power transfer circuit. The wireless activation of the powerless interior sensor system provides compatibility with multiple external power configurations, thereby enabling efficacious and continuous monitoring of the subject.

### RELATED APPLICATIONS

The present application claims benefit of priority under 35 U.S.C §119 (e) from U.S. Provisional Application Ser. No. 62/292,051, filed Feb. 5, 2016, entitled “Dentally-Based Concussion Sensor for Enhanced Detection of Brain Injuries and Related Method Thereof” and U.S. Provisional Application Ser. No. 62/336,899, filed May 16, 2016, entitled “Dentally-Based Concussion Sensor for Enhanced Detection of Brain Injuries and Related Method Thereof,” the disclosures of which are hereby incorporated by reference herein in their entirety.

### FIELD OF THE INVENTION

The present invention relates generally to systems and devices for the detection of brain injuries. More specifically, the present invention relates to a system for activating a passive tooth-mounted sensor to provide continuous and real-time head impact data.

### BACKGROUND

Concussions cause damage to neurological functioning due to rapid cranial acceleration, typically caused by trauma or injury. Though the immediate effects of head trauma are not always readily apparent, repeated head trauma can have significant long-term consequences if appropriate care is not given. Studies, for instance, have linked repeated upper body injuries with higher rates of chronic traumatic encephalopathy (CTE)—a degenerative disease that targets healthy brain tissue. Onset is gradual, often years or decades, which makes this condition difficult to detect and treat. Worse yet are the long-term effects of concussion in children, as they can potentially lead to developmental issues, altering the growth of emotions and behavior.

Traditionally, the diagnosis of concussions was based on subjective interpretations of symptoms by a clinician. The subjective nature of these clinician evaluations led many head injuries to be misclassified or undiagnosed. Although objective tests have now been developed to compare pre-impact baseline functioning with post-impact cognitive awareness, the subjectivity undermining the efficacy of clinician evaluations persists. Rather than being eliminated by objective cognitive awareness testing, this subjectivity has merely been injected into the detection of those who have suffered significant cranial impact requiring cognitive impairment testing.

Currently, detection is based on subjective observation by referees, sideline clinicians, and other third-party spotters. Such subjective observation will inevitably be under-inclusive, resulting in significant head trauma being undiagnosed and untreated. Thus, a strong need exists for an objective system for the detection of rapid cranial acceleration. In its most accurate form, such a detection system would comprise a sensor embedded in the rigid outer-shell of the skull, exposing the sensor to the same physical forces experienced by the brain. The invasiveness and complexity of such a system, however, renders it impracticable. Efforts to develop detection systems have therefore been constrained to developing externally mounted systems.

There are three main strategies currently being used to measure cranial acceleration, each approach positioning sensors at different locations. One representative system comprises mounting an accelerometer on an adhesive patch fastened to the skin behind a subject's ear. For the adhesive patch system, the coupling between the accelerometer on the patch and the subject's cranium is dependent upon the subject's skin elasticity as well as the extent of adhesion achieved. Evidence indicates that the adhesive skin patch experiences an average displacement of 3 millimeters with a standard deviation of 0.7 millimeters. With these critical parameters varying among individuals, a uniform transfer function to convert the accelerometer measurements into usable cranial acceleration data is impossible to achieve.

A second representative system comprises mounting an accelerometer to the inside of an American football helmet, which is to be worn by the subject during athletic competition. The helmet mounted system similarly fails to achieve uniform coupling, as the coupling is dependent upon factors such as the length of the individual's hair and how tightly the subject fastens the helmet. Evidence suggests that the helmet sensors experienced an average displacement of 5 millimeters with a standard deviation of 3 millimeters. Another disclosed system comprises an accelerometer mounted on a mouthpiece. Like the helmet mounted system and the adhesive patch system, the mouthpiece system fails to achieve sufficient coupling in the event that the subject is not firmly clamping down on the mouthpiece at the moment of impact.

In general, the fatal shortcoming of the existing systems is the weak coupling of the accelerometer to the subject's cranium. In other words, the existing technology fails to achieve the tight, secure attachment that is required. This shortcoming results in a disconnect between the measured and applied forces. Further analysis, then, cannot draw relationships, form conclusions, or aid in diagnosis.

In light of the above, a need arises for a system providing continuous and real-time monitoring and detection of rapid cranial acceleration and the resulting head trauma.

### Overview

An aspect of an embodiment of the present invention solves the problem of, among other things, weak coupling currently rendering prior art systems inadequate. Firmly secured to the skull through periodontal ligaments, teeth lack the ability to move or rotate independent of the skeletal systems. Unlike helmets or skin patches, they will remain fixated with respect to the skull despite a sudden force. Thus, the present invention will further biomedical understanding of multiple traumatic brain injuries, as well as aid in their rapid diagnosis and treatment. In various aspects, an embodiment of the present invention comprises, but is not limited thereto, a compact, dentally-based head impact detection system for enhanced detection of mild traumatic brain injuries. This system enables the collection of real-time, accurate and reproducible biomedical data while enabling continuous monitoring of a subject.

According to an aspect of an embodiment of the present invention, the continuous and real-time detection system is configured for use with an external power source to detect the head impact experienced by a subject. The system may comprise: a tooth-mounted passive sensor array configured to provide continuous and real-time impact data; a wireless power transfer circuit configured to transfer continuous power from an external power source to the tooth-mounted passive sensor array; and one or more processors configured to receive impact data measured at the tooth and extrapolate the impact experienced by the skull. In utilizing a tooth-mounted sensor, the system achieves biofidelity by using the rigid coupling between the subject's tooth and cranium. With the coupling between the subject's tooth and cranium established, the system multiplies the impact measurement provided by the tooth-mounted sensor by a uniform transfer function to extrapolate the impact experienced by the cranium. The uniform transfer function is determined using the standard spring and damper transfer function with inventor's comparative experimentation with sensors and providing values for the natural frequency and damping coefficient. In one aspect of an embodiment, the natural frequency is 96 Hertz and the damping coefficient is 11.7 Newton seconds per meter.

Further, the use of a passive sensor enables the system to maintain biofidelity while allowing continuous wear on a subject's tooth. To power the passive sensor, an aspect of an embodiment of the present invention comprises activating the system by powering the passive sensor using a wireless power transfer circuit. In one embodiment, the wireless power transfer circuit comprises an inductive power transfer circuit. In another embodiment, the wireless power transfer circuit comprises an ultrasound power transfer circuit.

According to an aspect of an embodiment of the invention, the local interior tooth-mounted passive detection system is configured for use with an external power source to provide continuous and real-time detection of head impact of a subject and may comprise: a dentally mountable passive sensor device; a wireless power receiver; said wireless power receiver configured to receive continuous power from the external power source; and said mountable dental sensor device configured to provide continuous and real-time impact data. In one embodiment, the dentally mountable sensor is configured to communicate, with one or more processors, the impact data measured at the subject's tooth and extrapolate the impact experienced by the subject's skull. The transmitted data is to be received by external processors for data processing and communication.

In another embodiment, the dentally mountable sensor device is provided with dental hardware, including but not limited to a dental band, configured to mount the dental sensor on the subject's molar. In a further embodiment, the dentally mountable sensor device is provided with a receiver configured to receive signals from a wireless power transmitter to define a transfer circuit configured to provide continuous power to the sensor device.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 schematically illustrates a side view of an aspect of an embodiment of the system according to the present disclosure.

FIG. 2 schematically illustrates a bottom view of an aspect of an embodiment of the system according to the present disclosure.

FIG. 3 schematically illustrates a side view of an aspect of an embodiment of the system in the context of an American football helmet containing the external power source and power transfer circuit.

FIG. 4 schematically illustrates a side view of an aspect of an embodiment of the local interior tooth-mounted system according to the present disclosure.

FIG. 5 schematically illustrates a side view of a subject's head experiencing rotation associated with cranial impact.

FIG. 6 schematically illustrates a front view of a subject's head experiencing rotation associated with cranial impact.

FIG. 7 schematically illustrates a top view of a subject's head experiencing rotation associated with cranial impact.

FIG. 8 schematically illustrates a side view of a subject's head experiencing positive linear movement along the x-axis, associated with cranial impact.

FIG. 9 schematically illustrates a side view of a subject's head experiencing negative linear movement along the x-axis, associated with cranial impact.

FIG. 10 schematically illustrates a side view of a subject's head experiencing positive linear movement along the y-axis, associated with cranial impact.

FIG. 11 schematically illustrates a side view of a subject's head experiencing positive linear movement along the y-axis, associated with cranial impact.

FIG. 12 schematically illustrates a the spring and damper model used to derive the transfer function between the subject's tooth and cranium utilized in an aspect of an embodiment of the present disclosure.

FIG. 13 schematically illustrates a perspective view of an aspect of an embodiment of the local interior sensor system.

FIG. 14 is a side view of an aspect of an embodiment of a schematic for the inductive power transfer circuit.

FIG. 15 is a block diagram illustrating an example of a machine upon which one or more aspects of embodiments of the present invention can be implemented.

FIG. 16 schematically illustrates a perspective view of an aspect of an embodiment of the system in the context of a military helmet containing the external power source and power transfer circuit.

FIG. 17 schematically illustrates a perspective view of an aspect of an embodiment of the system in the context of a motorcycle helmet containing the external power source and power transfer circuit.

FIG. 18 schematically illustrates a perspective view of an aspect of an embodiment of the system in the context of an American football helmet containing the external power source and power transfer circuit.

FIG. 19 schematically illustrates a perspective view of an aspect of an embodiment of the system in the context of at least one ear piece containing the external power source and power transfer circuit.

FIG. 20 schematically illustrates a perspective view of an aspect of an embodiment of the system in the context of a necklace containing the external power source and power transfer circuit.

### DETAILED DESCRIPTION OF EXEMPLARY EMBODIMENTS

Turning now to the drawings, the invention comprises various systems to more accurately measure head impact and thereby detect potential head trauma. An aspect of an embodiment of the present invention provides, but is not limited thereto, a system for monitoring the impact measured at a subject's tooth and extrapolating that tooth impact data to determine the impact experienced by the subject's cranium. These impact detection measurements are critical to a wide range of applications including concussion detection in the short term and the development of a greater understanding of multiple traumatic brain injuries in the long term.

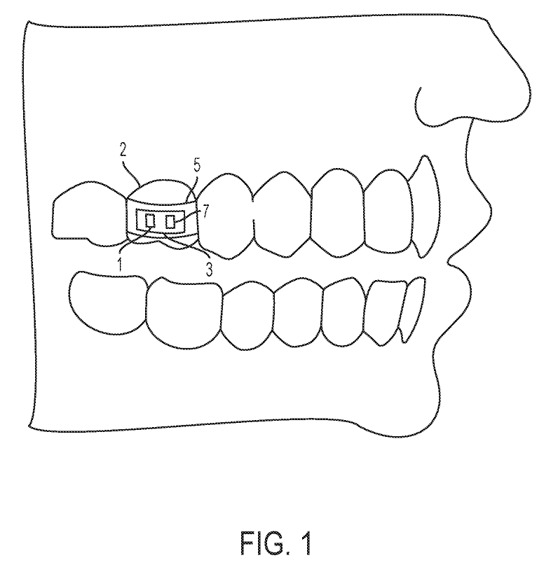

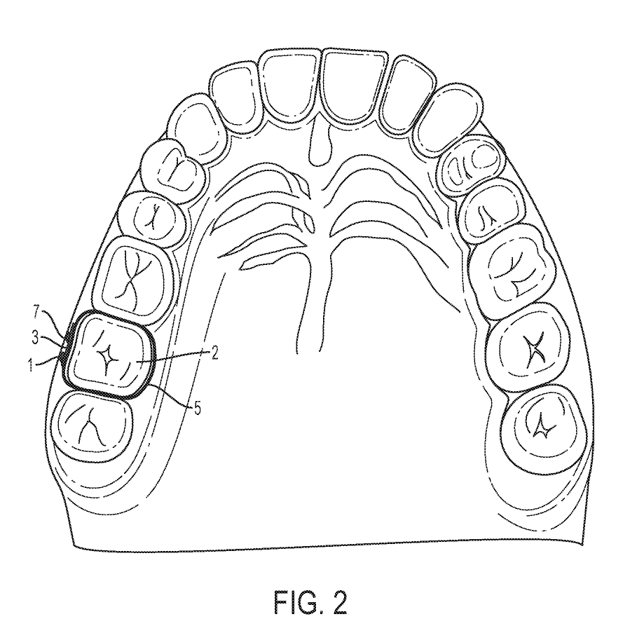

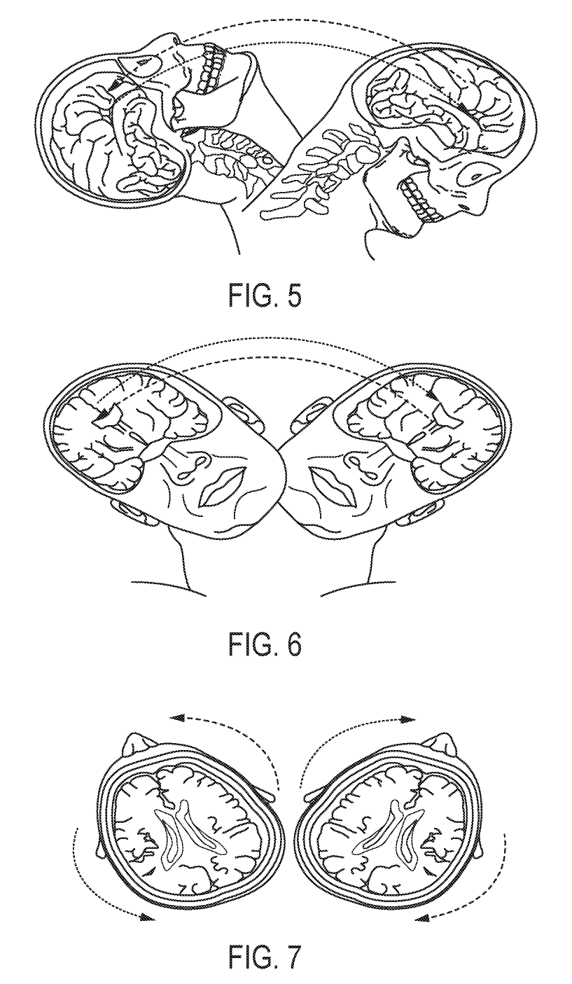

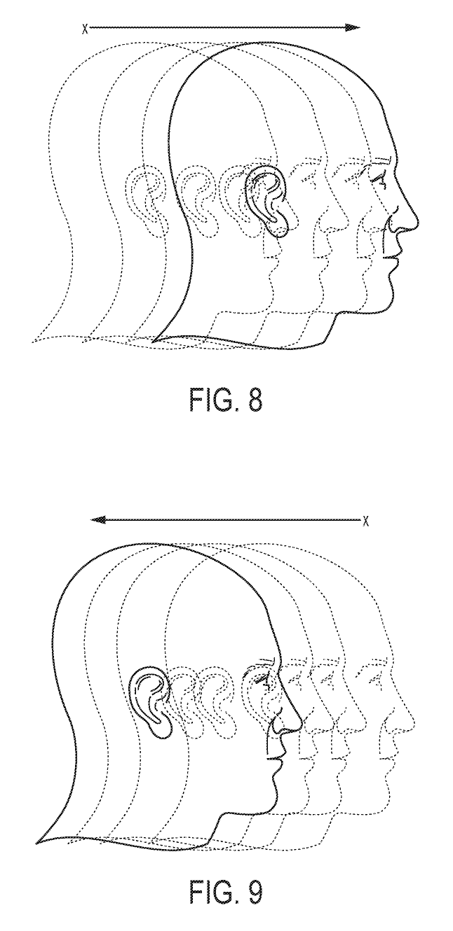

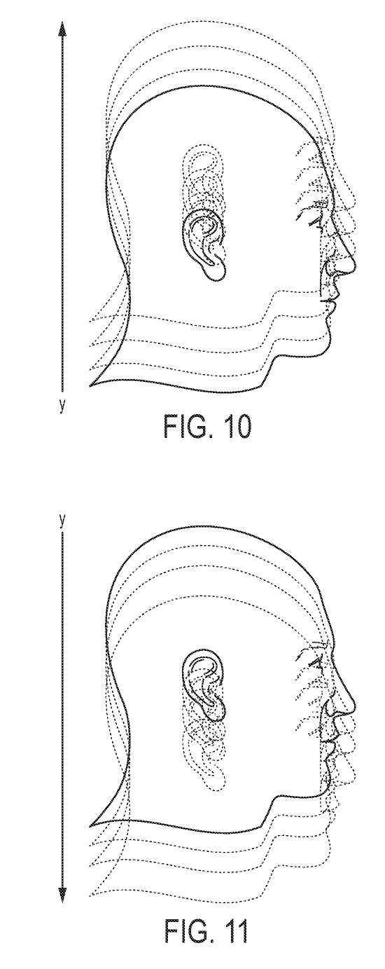

The present invention detects the head impact of a subject using a tooth-mounted passive sensor configured to provide continuous and real-time impact data. As depicted in FIGS. 1, 2, and 13, an aspect of an embodiment of the present invention includes a local interior tooth-mounted sensor system 8. The local interior tooth-mounted sensor system 8 comprises a detection unit 3 and dental hardware 5 to mount the detection unit to the subject's tooth 2. In one embodiment, the tooth 2 is one of the subject's upper molar. As disclosed, the detection unit 3 further comprises a passive sensor array 1 and a receiver unit 7. In one embodiment, the passive sensor array 1 is an accelerometer array. The accelerometer array may comprise three rotational accelerometers configured to measure the rotational acceleration of the subject's head along the three axes as depicted in FIGS. 5-7. In a separate embodiment, the accelerometer array can comprise three linear accelerometers configured to measure the linear acceleration of the subject's head along the two axes (x- and y) depicted in FIGS. 8-11, and the linear acceleration along the z-axis (not shown). In one embodiment, the accelerometer array comprises three linear accelerometers and three rotational accelerometers configured to measure both the linear acceleration along all three axes and the rotational acceleration about all three axes. In a related embodiment, the sensor array 1 comprises at least one ADXL375 accelerometer.

In an embodiment depicted in FIG. 13, the local interior sensor system 8 further comprises a printed circuit board 4 and microprocessor 6. The printed circuit board 4 provides the electrical connections between the microprocessor 6, passive tooth-mounted sensor 1, and the wireless power receiver 7. The microprocessor 6 is configured to communicate the tooth impact data measured by the sensor 1 to at least one external processor, such as the processor 402 depicted in FIG. 15. In order for the tooth impact data measured at the tooth to aid in the detection of head traumas, the data is processed by the at least one external processor, such as the processor 402 depicted to extrapolate the impact experienced by the subject's cranium. As disclosed, this extrapolation occurs by multiplying the impact data measured at the tooth by a transfer function.

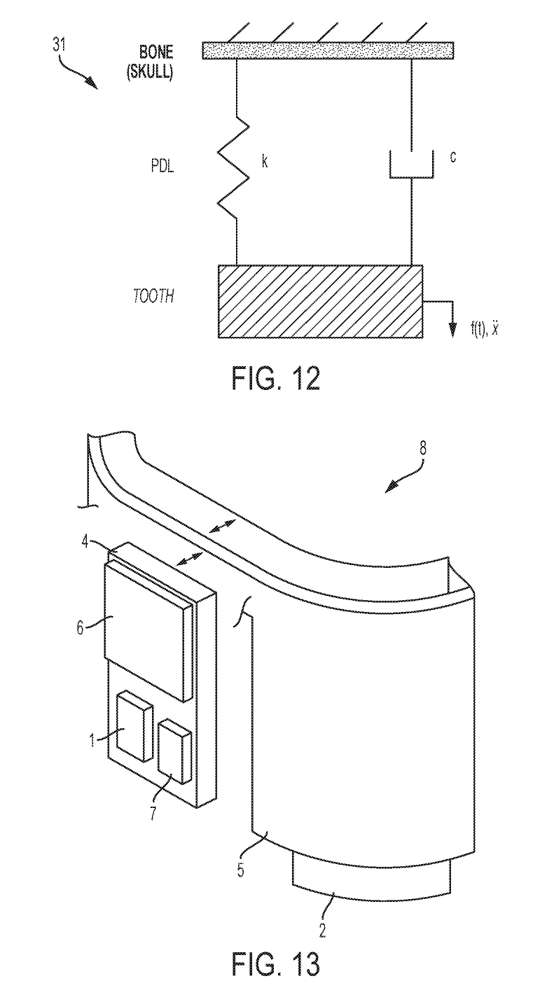

Mounting the sensor array 1 to the subject's tooth using dental hardware 5 achieves firm and uniform coupling. In one embodiment, depicted in FIGS. 1, 2, and 13, the dental hardware 5 consists of a dental band. In the alternative, the dental hardware 5 comprises adhesive such as that used in mounting orthodontic braces to teeth. With uniform coupling established, a transfer function can be established by modeling the periodontal ligaments securing the tooth to the skull using the spring and damper model 31 depicted in FIG. 12. The Laplace transfer function corresponding to such a model is:

$$T ( s ) = \frac{1}{\frac{{s}^{2}}{{ω}_{n}} + \frac{2 ζ s}{{ω}_{n}} + 1} \qquad ( Equation 1 )$$

wherein ωₙ is the natural frequency and ζ is the damping coefficient. Through the inventors' laboratory experimentation with the skull embedded sensors and tooth-mounted sensors, the inventors have established that in one embodiment, the natural frequency for this transfer function is 96 Hertz and the damping coefficient is 11.7 Newton seconds per meter. In this embodiment, the transfer function is as follows:

$$T ( s ) = \frac{1}{\frac{{s}^{2}}{96} + \frac{2 \; 3.4 s}{96} + 1} \qquad ( Equation 2 )$$

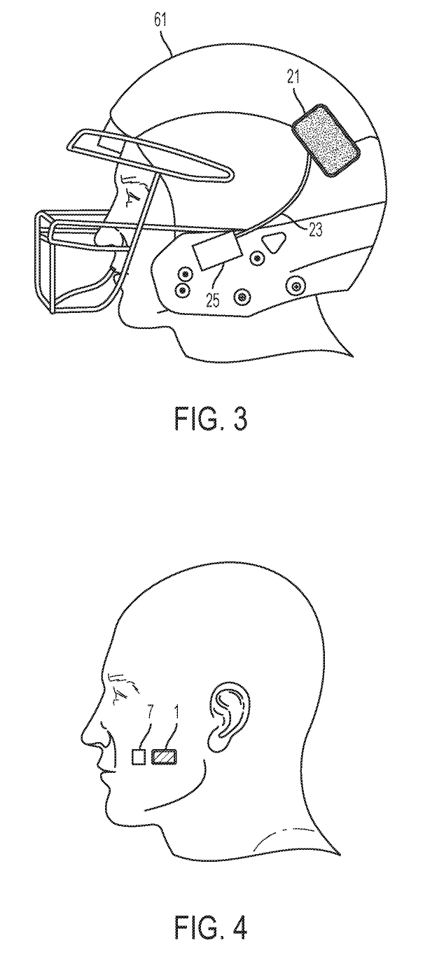

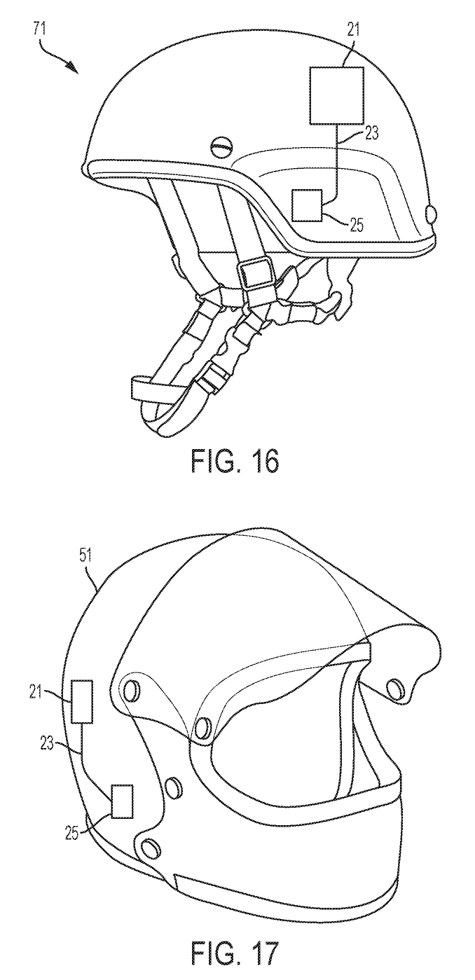

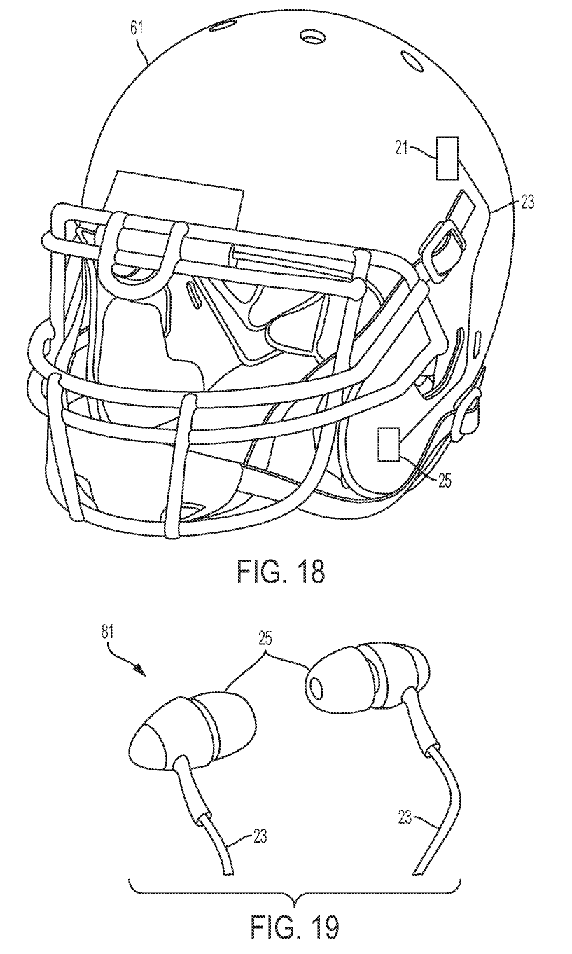

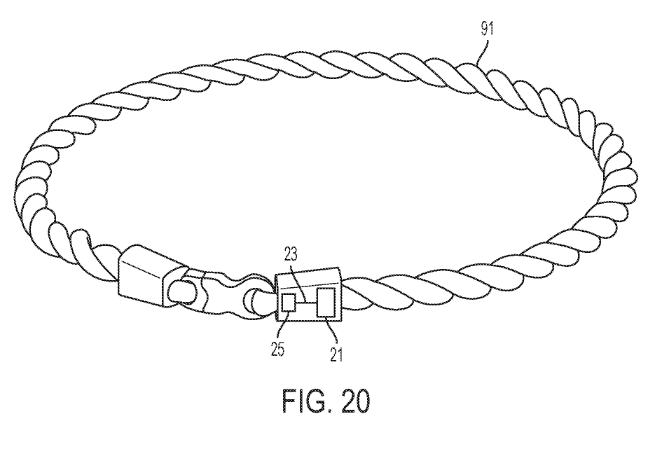

In an aspect of an embodiment of the present invention, the head impact detection system further comprises a wireless power transfer circuit to provide continuous power to the passive sensor array 1 mounted on the subject's tooth 2. To enable continuous wear of the local interior sensor system 8, the transfer circuit is configured to transmit power from an external power source 21 located outside of the subject's mouth to the receiver located in the subject's mouth as depicted in FIG. 4. The external power source 21 may comprise either an alternating current source or a direct current source. As depicted in FIGS. 3 and 16-18, the external power source 21 may be disposed in mechanical connection with a helmet. This helmet may comprise, but is not limited to, an American football helmet 61 as in FIGS. 3 and 18, a motorcycle helmet 51 as in FIG. 17, or a military helmet 71 as in FIG. 16. Alternatively, the external battery source 21 may be disposed in mechanical connection with at least one ear piece 81 as depicted in FIG. 19 or a necklace 91 as in FIG. 20. In one embodiment, the ear piece 81 comprises at least one ear bud. The external power source 21 delivers power to the passive tooth-mounted sensor array 1 through the use of a wireless power transfer circuit. Alternatively, the external battery source 21 may be disposed in mechanical connection with any equipment, accessory or apparel that may be worn or used by the subject.

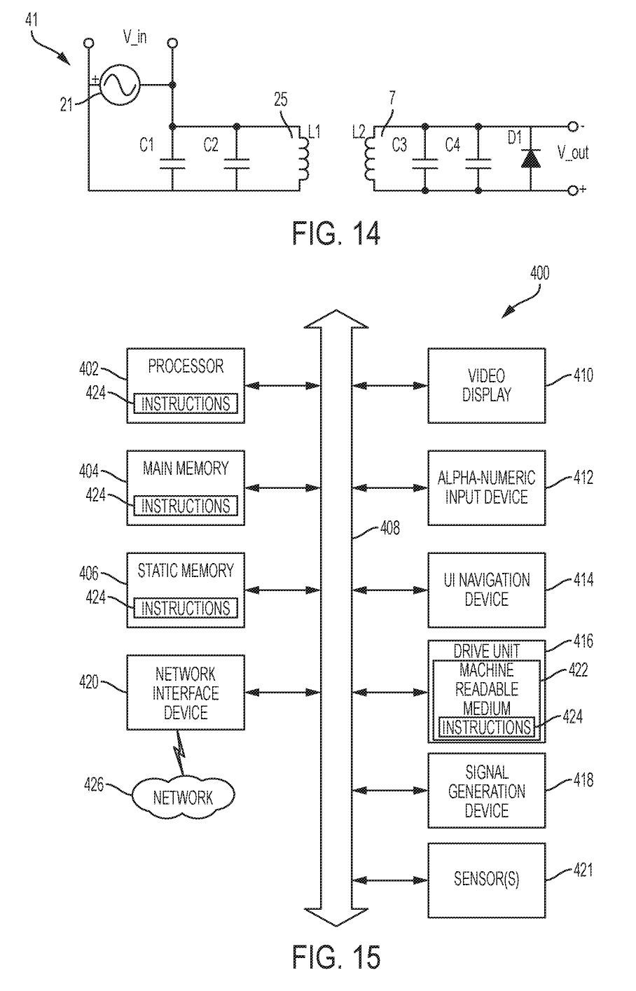

In general, the wireless power transfer circuit comprises a wireless power transmitter 25 and a wireless power receiver 7. In one embodiment, the wireless power transfer circuit comprises an inductive power transfer circuit 41 as depicted in FIG. 14, for example. The inductive power transfer circuit 41 is configured to pass an alternating current through a primary coil, represented by inductive coil L1 in FIG. 14, to induce the generation of a cycling magnetic field which is subsequently, in the presence of a secondary coil, represented by inductive coil L2 in FIG. 14, converted into a usable electrical signal for powering said tooth-mounted passive sensor. In the inductive power transfer circuit, the primary coil represents the wireless power transmitter 25 and the secondary coil represents the wireless power receiver 7.

However, in an alternative embodiment, the wireless power transfer circuit comprises an ultrasound power transfer circuit (rather than the aforementioned inductive power transfer circuit). Regarding the ultrasound transfer circuit, the ultrasound power transfer circuit comprises an ultrasound transmitter and an ultrasound receiver. For example, the ultrasound power transfer circuit may be configured to transfer power wirelessly from an ultrasound transmitter to an ultrasound receiver for powering the tooth-mounted passive sensor. Accordingly, in the ultrasound power transfer circuit embodiment, the ultrasound transmitter represents the wireless power transmitter 25 and the ultrasound receiver represents the wireless power receiver 7.

As depicted in FIGS. 16-20, the external power source 21 is connected to the wireless power transmitter 25 through a connecting wire 23.

FIG. 15 is a block diagram illustrating an example of a machine upon which one or more aspects of embodiments of the present invention can be implemented.

FIG. 15 illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Any of the components or modules referred to with regards to any of the present invention embodiments of the device discussed herein, may be integrally or separately formed with one another. Further, redundant functions or structures of the components or modules may be implemented.

Any of the components or modules may be a variety of widths and lengths as desired or required for operational purposes.

It should be appreciated that various sizes, dimensions, contours, rigidity, shapes, flexibility and materials of any of the components or portions of components in the various embodiments of the device discussed throughout may be varied and utilized as desired or required. Similarly, locations and alignments of the various components may vary as desired or required. Moreover, modes and mechanisms for connectivity or interchangeability may vary.

It should be appreciated that the device and related components of the device discussed herein may take on all shapes along the entire continual geometric spectrum of manipulation of x, y and z planes to provide and meet the environmental, and structural demands and operational requirements. Moreover, locations, connections and alignments of the various components may vary as desired or required.

### EXAMPLES

Practice of an aspect of an embodiment (or embodiments) of the invention will be still more fully understood from the following examples and experimental results, which are presented herein for illustration only and should not be construed as limiting the invention in any way.

### Example No. 1: Quantitative Constraints/Specifications

TABLE 1

| Need | Design | Unit of | Marginal | Ideal |
|---|---|---|---|---|
| # | Constraint/ | Measure | (Acceptable) | Value |
| 1 | A. Translational | A. m/s² | A. 80-99 | A. ≧100 |
| | Acceleration | B. rad/s² | B. 5900-7900 | B. ≧6000 |
| | B. Rotational | | | |
| | Acceleration | | | |
| 2 | Coupling Capacity | Netwons · mm | 200-270 | 235 |
| 3 | Sampling Frequency | Hz | 1 kHz | ≧1 kHz |
| 4 | Inductive | Volts (DC) | 2-3.6 | 3.3 |
| | Power | | | |
| | Transfer | | | |
| 5 | External power | Volts (DC) | 9 | 9 |
| | source | | | |
| 6 | Wireless Data | kilobits/s | 400 | ≧400 |
| | Transmission | | | |
| 7 | Radio | Yards | 104 | 200 |
| | Transmission | | | |
| | Distance | | | |
| 8 | System Battery Life | Minutes | 192 | 240 |

### Example No. 2: Exemplary Components

TABLE 2
Item

Arduino Boards & Atmel microcontrollers
Inductive Power Circuitry
Secondhand Football Helmetto Mount Circuitry into
Xbee Radios
Tooth mounted sensor packs
Bluetooth Modules
Mouthpiece Molding/3D Printing
Lithium Polymer Battery Charges (They malfunction if
chargedwrong)
Lithium Polymer Battery
Belt to hold electronics
Big breadboard
Temperature controlled soldering iron
Consumables (solder, flux, duct tape, etc.)
Wires and wire strippers
Dental Implant Teeth Model
Hawley Retainer Model

### ADDITIONAL EXAMPLES

### Example 1

A continuous and real-time detection system for detecting the head impact of a subject, for use with an external power source The system may comprise: a tooth-mounted passive sensor array configured to provide continuous and real-time impact data; a wireless power transfer circuit configured to transfer continuous power from said external power source to said tooth-mounted passive sensor array; and one or more processors configured to receive said impact data measured at the tooth and extrapolate the impact experienced by the skull.

### Example 2

The system of example 1, wherein said extrapolation is achieved by multiplying said impact data measured at the tooth by a transfer function.

### Example 3

The system of example 2, wherein said transfer function is defined as:

$$T ( s ) = \frac{1}{\frac{{s}^{2}}{{ω}_{n}} + \frac{2 ζ s}{{ω}_{n}} + 1}$$

wherein ωₙ is the natural frequency and ζ is the damping coefficient.

### Example 4

The system of example 3, wherein the natural frequency is 96 Hertz.

### Example 5

The system of example 3 (as well as subject matter in whole or in part of example 4), wherein the damping coefficient is 11.7 Newton seconds per meter.

### Example 6

The system of example 1 (as well as subject matter of one or more of any combination of examples 2-5, in whole or in part), further comprising an external power source.

### Example 7

The system of example 1 (as well as subject matter of one or more of any combination of examples 2-6, in whole or in part), wherein said tooth-mounted passive sensor array is an accelerometer array.

### Example 8

The system of example 7, wherein said accelerometer array comprises three linear accelerometers and three rotational accelerometers, said array capable of measuring rotational and linear acceleration.

### Example 9

The system of example 7 (as well as subject matter in whole or in part of example 8), wherein said accelerometer array comprises any combination of at least one linear or rotational accelerometer, said array capable of measuring rotational or linear acceleration.

### Example 10

The system of example 7 (as well as subject matter in whole or in part of example 9), wherein said accelerometer array comprises three rotational accelerometers, said array capable of measuring only rotational acceleration.

### Example 11

The system of example 7 (as well as subject matter in whole or in part of example 10), wherein said accelerometer array comprises three linear accelerometers, said array capable of measuring only linear acceleration.

### Example 12

The system of example 1 (as well as subject matter of one or more of any combination of examples 2-11, in whole or in part), wherein said wireless power transfer circuit comprises an inductive power transfer circuit configured to pass an alternating current through a primary coil to induce the generation of a cycling magnetic field which is subsequently, in the presence of a secondary coil, converted into a usable electrical signal for powering said tooth-mounted passive sensor.

### Example 13

The system of example 12, wherein said secondary coil is configured to be securely mounted in mechanical communication with the molar of the subject.

### Example 14

The system of example 12 (as well as subject matter in whole or in part of example 13), further comprising a helmet wherein said primary coil is disposed in mechanical communication with the helmet.

### Example 15

The system of example 14, wherein said helmet comprises a military helmet, motorcycle helmet, or American football helmet.

### Example 16

The system of example 12 (as well as subject matter in whole or in part of example 15), further comprising at least one ear piece, wherein said primary coil is disposed in mechanical communication with the at least one ear piece.

### Example 17

The system of example 12 (as well as subject matter of one or more of any combination of examples 2-11 and 13-16, in whole or in part), further comprising a necklace, wherein said primary coil is disposed in mechanical communication with the necklace.

### Example 18

The system of example 1 (as well as subject matter of one or more of any combination of examples 2-11 and 13-17, in whole or in part), wherein said wireless power transfer circuit comprises an ultrasound power transfer circuit configured to transfer power wirelessly from an ultrasound transmitter to an ultrasound receiver.

### Example 19

The system of example 18, wherein said ultrasound receiver is configured to be securely mounted in mechanical communication with the molar of the subject.

### Example 20

The system of example 19, further comprising a helmet, wherein said ultrasound transmitter is disposed in mechanical communication with the helmet.

### Example 21

The system of example 20, wherein the helmet comprises a military helmet, motorcycle helmet, or American football helmet.

### Example 22

The system of example 18 (as well as subject matter in whole or in part of example 21), further comprising at least one ear piece, wherein the ultrasound transmitter is disposed in mechanical communication with the at least one ear piece.

### Example 23

The system of example 18 (as well as subject matter in whole or in part of example 22), further comprising a necklace, wherein the ultrasound transmitter is disposed in mechanical communication with the necklace.

### Example 24

The system of example 1 (as well as subject matter of one or more of any combination of examples 2-23, in whole or in part), wherein the tooth-mounted passive sensor array is removably affixed through orthodontic methods to the subject's molar.

### Example 25

A tooth-mounted passive detection system for continuous and real-time detection of the head impact of a subject, for use with an external power source. The sensor system may comprise: a dentally mountable passive sensor device and a wireless power receiver. The wireless power receiver may be configured receive continuous power from the external power source and the mountable dental sensor device may be configured to provide continuous and real-time impact data.

### Example 26

The system of example 25, wherein said dentally mountable sensor is configured to communicate with one or more processors said impact data measured at the subject's tooth and extrapolate the impact experienced by the subject's skull.

### Example 27

The system of example 25 (as well as subject matter in whole or in part of example 26), wherein said dentally mountable sensor device is an accelerometer array.

### Example 28

The system of example 25 (as well as subject matter in whole or in part of example 27), wherein said dentally mountable sensor device is provided with dental hardware configured to mount said dental sensor to be disposed on a molar.

### Example 29

The system of example 28, wherein said dental hardware comprises a dental band.

### Example 30

The system of example 25 (as well as subject matter in whole or in part of example 29), wherein said dentally mountable sensor device is provided with a receiver configured to receive signals from said wireless power transmitter to define a transfer circuit that is configured to provide the continuous power to said dentally mountable sensor device.

### Example 31

The system of example 25 (as well as subject matter in whole or in part of example 30), wherein said dentally mountable sensor device is provided with said one or more processors.

### Example 32

The system of example 25 (as well as subject matter in whole or in part of example 31), further comprising: one or more processors provided for use with said dentally mountable sensor device.

### Example 33

The system of example 25 (as well as subject matter in whole or in part of example 32), further comprising: an external power source provided for use with said dentally mountable sensor device.

### Example 34

The method of using any of the devices, systems, assemblies, or their components provided in any one or more of examples 1-33.

### Example 35

The method of manufacturing any of the devices, systems, assemblies, or their components provided in any one or more of examples 1-33.

### Example 36

It is noted that the machine readable medium or computer useable medium may be configured to execute the subject matter pertaining to system or related methods disclosed in examples 1-33.

### REFERENCES

The devices, systems, apparatuses, materials, components, computer readable medium, algorithms, and methods of various embodiments of the invention disclosed herein may utilize aspects disclosed in the following references, applications, publications and patents and which are hereby incorporated by reference herein in their entirety (and which are not admitted to be prior art with respect to the present invention by inclusion in this section):
1. Christopher, J. J., Sochor, M. R., Pellettiere, J., & Salzar, R. S. (2013). Assessment of Ear- and Tooth-Mounted Accelerometers as Representative of Human Head Response (SAE Technical Paper No. 2013-01-0805). Warrendale, Pa.: SAE Technical Paper. Retrieved from http://papers.sae.org/2013-01-0805/2.
Cicciù, M., Bramanti, E., Signorino, F., Cicciù, A., & Sortino, F. (2013). Experimental Study on Strength Evaluation Applied for Teeth Extraction: An In Vivo Study. The Open Dentistry Journal, 7, 20-26. http://doi.org/10.2174/1874210601307010020
3. Daneshvar, D. H., Riley, D. O., Nowinski, C. J., McKee, A. C., Stern, R. A., & Cantu, R. C. (2011). Long Term Consequences: Effects on Normal Development Profile after Concussion. Physical Medicine and Rehabilitation Clinics of North America, 22(4), 683-700. http://doi.org/10.1016/j.pmr.2011.08.009
4. FITGuard by Force Impact Technologies. (n.d.). Retrieved Nov. 22, 2015, fromhttps://www.fitguard.me/5.
Flint, J. (2012, Sep. 18). NFL games are getting longer. Los Angeles Times. Retrieved from http://articles.latimes.com/2012/sep/18/entertainment/la-et-ct-nflrun-20120918
6. Foster, T. (2012, Dec. 18). The Helmet That Can Save Football. Retrieved Dec. 1, 2015, from http://www.popsci.com/science/article/2013-08/helmet-wars-and-new-helmet-could-protect-us-all
7. Hanna, J., Goldschmidt, D., & Flower, K. (2015, Oct. 11). Many ex-NFL players had brain disease linked to concussions—CNN.com. Retrieved Dec. 1, 2015, from http://www.cnn.com/2015/09/18/health/nfl-brain-study-cte/index.html
8. Jadischke, R., Viano, D. C., Dau, N., King, A. I., & McCarthy, J. (2013). On the accuracy of the Head Impact Telemetry (HIT) System used in football helmets. Journal of Biomechanics, 46(13), 2310-2315. http://doi.org/10.1016/j.jbiomech. 2013.05.030
9. Linendoll, K. (2013, Jan. 7). Could X2's skin patch detect concussions? Retrieved Dec. 1, 2015, from http://espn.go.com/blog/playbook/tech/post/_/id/3547
10. McKinlay A, Dalrymple-Alford J C, Horwood L J, Fergusson D M. Long term psychosocial outcomes after mild head injury in early childhood. J Neurol Neurosurg Psychiatry. 2002 September; 73(3):281-288.
11. NFL reminds clubs of sidelines' policy. (n.d.). Retrieved Sep. 28, 2015, from http://www.nfl.com/news/story/09000d5d81cfb490/article/nfl-reminds-clubs-of-sidelines-policy
12. Nordqvist, J. (2013, Feb. 18). Concussions Cause Long-Term Effects Lasting Decades. Retrieved Nov. 18, 2015, from http://www.medicalnewstoday.com/articles/256518.php
13. Remington, A. (2014, Oct. 22). Sports-related concussions and traumatic brain injuries: Research roundup. Retrieved from http://journalistsresource.org/studies/society/public-health/sports-related-concussions-head-injuries-what-does-research-say
14. Wu, L., Nangia, V., Bui, K., Hammoor, B., Kurt, M., Hernandez, F., . . . Camarillo, D. (2015). In Vivo Evaluation of Wearable Head Impact Sensors. Annals of Biomedical Engineering. http://doi.org/10.1007/s10439-015-1423-3
15. Zhang L, Yang K H, King A I. A Proposed Injury Threshold for Mild Traumatic Brain Injury. J. Biomech. Eng. 2004; 126(2):226-236.
16. U.S. Patent Application Publication No. US 2011/0181418 A1, Mack, et al., “Communication System for Impact Sensors”, Jul. 28, 2011
17. U.S. Pat. No. 6,941,952 B1, Rush, III, “Athletic Mouthpiece Capable of Sensing Linear and Rotational Forces and Protective Headgear for use with the Same”, Sep. 13, 2005.
18. U.S. Patent Application Publication No. US 2011/0184319 A1, Mack, et al., “Mouth Guard with Sensor”, Jul. 28, 2011.
19. U.S. Patent Application Publication No. US 2011/0184663 A1, Mack, et al., “Head Impact Analysis and Comparison System”, Jul. 28, 2011.
20. U.S. Patent Application Publication No. US 2011/0179851 A1, Mack, et al., “Mouth Guard Formation Methods”, Jul. 28, 2011.
21. U.S. Pat. No. 9,007,217 B1, Anvari, K., “Helmet with Patch Antennas to Detect, Prevent, and Minimize Head Concussion”, Apr. 14, 2015.
22. U.S. Pat. No. 8,947,195 B1, Anvari, K., “Helmet Impact Detection and Prevention Mechanism to Minimize Head Concussion”, Feb. 3, 2015.
23. U.S. Pat. No. 5,539,935, Rush, III, “Sports Helmet”, Jul. 30, 1996.
24. U.S. Pat. No. 5,621,922, Rush, III, “Sports Helmet Capable of Sensing Linear and Rotational Forces”, Apr. 22, 1997.
25. U.S. Patent Application Publication No. US 2013/0060168 A1, Chu, et al., “Systems and Methods for Monitoring a Physiological Parameter of Persons Engaged in Physical Activity”, Mar. 7, 2013.
26. U.S. Patent Application Publication No. US 2015/0238143 A1, Meurer, R., “Helmet Head Impact Tracking and Monitoring System”, Aug. 27, 2015.
27. U.S. Pat. No. 7,682,303 B2, Abolfathi, A., “Methods and Apparatus for Transmitting Vibrations”, Mar. 23, 2010.
28. U.S. Pat. No. 9,240,688 B2, Hwang, G. et al., “Ultrasonic Wireless Power Transmitter and Receiver Apparatuses, and Method for Wireless Charging Thereof”, Jan. 19, 2016.
29. U.S. Patent Application Publication No. US 2013/0271088 A1, Hwang, G., et al., “Ultrasonic Wireless Power Transmitter and Receiver Apparatuses, and Method for Wireless Charging Thereof”, Oct. 17, 2013.

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the present invention may have a variety of sizes, contours, shapes, compositions and materials as desired or required.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. A continuous and real-time detection system for detecting the head impact of a subject, for use with an external power source, said system comprising:
a tooth-mounted passive sensor array configured to provide continuous and real-time impact data;
a wireless power transfer circuit configured to transfer continuous power from said external power source to said tooth-mounted passive sensor array; and
one or more processors configured to receive said impact data measured at the tooth and extrapolate the impact experienced by the skull.

2. The system of claim 1, wherein said extrapolation is achieved by multiplying said impact data measured at the tooth by a transfer function.

3. The system of claim 2, wherein said transfer function is defined as:
$$T ( s ) = \frac{1}{\frac{{s}^{2}}{{ω}_{n}} + \frac{2 ζ s}{{ω}_{n}} + 1}$$
wherein ωₙ is the natural frequency and ζ is the damping coefficient.

4. The system of claim 3, wherein the natural frequency is 96 Hertz.

5. The system of claim 3, wherein the damping coefficient is 11.7 Newton seconds per meter.

6. The system of claim 1, further comprising an external power source.

7. The system of claim 1, wherein said tooth-mounted passive sensor array is an accelerometer array.

8. The system of claim 7, wherein said accelerometer array comprises three linear accelerometers and three rotational accelerometers, said array capable of measuring rotational and linear acceleration.

9. The system of claim 7, wherein said accelerometer array comprises any combination of at least one linear or rotational accelerometer, said array capable of measuring rotational or linear acceleration.

10. The system of claim 7, wherein said accelerometer array comprises three rotational accelerometers, said array capable of measuring only rotational acceleration.

11. The system of claim 7, wherein said accelerometer array comprises three linear accelerometers, said array capable of measuring only linear acceleration.

12. The system of claim 1, wherein said wireless power transfer circuit comprises an inductive power transfer circuit configured to pass an alternating current through a primary coil to induce the generation of a cycling magnetic field which is subsequently, in the presence of a secondary coil, converted into a usable electrical signal for powering said tooth-mounted passive sensor.

13. The system of claim 12, wherein said secondary coil is configured to be securely mounted in mechanical communication with the molar of the subject.

14. The system of claim 12, further comprising a helmet wherein said primary coil is disposed in mechanical communication with the helmet.

15. The system of claim 14, wherein said helmet comprises a military helmet, motorcycle helmet, or American football helmet.

16. The system of claim 12, further comprising at least one ear piece, wherein said primary coil is disposed in mechanical communication with the at least one ear piece.

17. The system of claim 12, further comprising a necklace, wherein said primary coil is disposed in mechanical communication with the necklace.

18. The system of claim 1, wherein said wireless power transfer circuit comprises an ultrasound power transfer circuit configured to transfer power wirelessly from an ultrasound transmitter to an ultrasound receiver.

19. The system of claim 18, wherein said ultrasound receiver is configured to be securely mounted in mechanical communication with the molar of the subject.

20. The system of claim 19, further comprising a helmet, wherein said ultrasound transmitter is disposed in mechanical communication with the helmet.

21. The system of claim 20, wherein the helmet comprises a military helmet, motorcycle helmet, or American football helmet.

22. The system of claim 18, further comprising at least one ear piece, wherein the ultrasound transmitter is disposed in mechanical communication with the at least one ear piece.

23. The system of claim 18, further comprising a necklace, wherein the ultrasound transmitter is disposed in mechanical communication with the necklace.

24. The system of claim 1, wherein the tooth-mounted passive sensor array is removably affixed through orthodontic methods to the subject's molar.

25. A tooth-mounted passive detection system for continuous and real-time detection of the head impact of a subject, for use with an external power source, said sensor system comprises:
a dentally mountable passive sensor device;
a wireless power receiver;
said wireless power receiver is configured receive continuous power from the external power source; and
said mountable dental sensor device is configured to provide continuous and real-time impact data.

26. The system of claim 25, wherein said dentally mountable sensor is configured to communicate with one or more processors said impact data measured at the subject's tooth and extrapolate the impact experienced by the subject's skull.

27. The system of claim 25, wherein said dentally mountable sensor device is an accelerometer array.

28. The system of claim 25, wherein said dentally mountable sensor device is provided with dental hardware configured to mount said dental sensor to be disposed on a molar.

29. The system of claim 28, wherein said dental hardware comprises a dental band.

30. The system of claim 25, wherein said dentally mountable sensor device is provided with a receiver configured to receive signals from said wireless power transmitter to define a transfer circuit that is configured to provide the continuous power to said dentally mountable sensor device.

31. The system of claim 25, wherein said dentally mountable sensor device is provided with said one or more processors.

32. The system of claim 25, further comprising:
one or more processors provided for use with said dentally mountable sensor device.

33. The system of claim 25, further comprising:
an external power source provided for use with said dentally mountable sensor device.
